(19) United States
(12) Patent Application Publication
(10) Pub. No.: US 2024/0366919 A1
(43) Pub. Date: Nov. 7, 2024

(54) DEVICE FOR THE APPLICATION OF A THERAPEUTIC SUBSTANCE IN THE FORM OF A FLUID

(71) Applicant: FUJIFILM CORPORATION, Tokyo (JP)

(72) Inventors: Johannes DIETER, Bamberg (DE); Matthias SCHRADER, Nürnberg (DE)

(21) Appl. No.: 18/651,068

(22) Filed: Apr. 30, 2024

(30) Foreign Application Priority Data
May 4, 2023 (EP) 23 171 665.5

(51) Int. Cl. A61M 31/00; A61M 25/00

(52) U.S. Cl. CPC A61M 31/002; A61M 25/0082; A61M 2209/08; A61M 2210/1064

(57) ABSTRACT

A device for applying a fluid therapeutic substance to a tissue wall which surrounds a body cavity. The device is arranged on a catheter that is insertable into the body cavity and, after insertion, can be expanded radially relative to the catheter to touch the tissue wall, the fluid supplied via the catheter reaching the tissue wall via outlet openings. The catheter is designed as a tube of an endoscopic device, the tube receiving the device at its distal end and having a lumen in which a mechanical control element extends. By longitudinal movements of the control element, which are generated proximally via an actuating element, the device is adjusted between a state of smallest radial extension and a state of largest radial extension, whereby it has the outlet openings for the fluid at least in its section which touches the tissue wall in the spread-open state.

### CROSS-REFERENCE TO RELATED APPLICATIONS

The present application claims priority of EP 23 171 665.5, filed May 4, 2023, the priority of this application is hereby claimed, and this application is incorporated herein by reference.

### BACKGROUND OF THE INVENTION

The invention relates to a device for applying a therapeutic substance in the form of a fluid to a tissue wall which surrounds a body cavity, wherein the device is arranged on a catheter which can be inserted into the body cavity and, after insertion, can be expanded radially with respect to the catheter to touch the tissue wall, and wherein the fluid supplied via the catheter is directed to the tissue wall via fluid outlet openings.

For an effective treatment of certain diseases that occur in body cavities and organs, it may be appropriate to apply therapeutic substances to the tissue affected by the disease, i.e. a tissue wall surrounding the body cavity or an organ adjacent to it. In this way, the substance, which is often unsuitable for oral administration due to side effects, can be introduced directly and specifically into the diseased tissue in order to treat it.

Another medical problem in which a tissue wall of a body cavity needs to be treated is gastrointestinal bleeding, which occurs with a high incidence as acute ulcer bleeding in the upper gastrointestinal tract. These require immediate and rapid treatment. As part of a haemostasis therapy in which surgical intervention should be avoided as far as possible. Endoscopic devices are often used for this purpose, with which medical clips are placed in the tissue wall to close the wound and thus stop the bleeding. Severe bleeding can occur in the gastrointestinal tract, particularly after endoscopic resection of tissue, such as after a polypectomy. Furthermore, bleeding in the human body can also occur in the lower gastrointestinal tract, which can also be stopped by means of endoscopically applied medical clips, whereby this is done as part of a colonoscopy. Overall, the use of endoscopic therapy results in a low recurrent bleeding rate, provided it is not a case of peracute bleeding with massive activity.

Furthermore, in addition to the use of clips, injection therapy or thermotherapy can also be used for endoscopic haemostasis. In thermotherapy, thermal means are used to stop the bleeding, such as cauterizing one or more points in the tissue. This can damage the surrounding parenchymal tissue.

A device for applying a therapeutic substance, which is in the form of a fluid, to a tissue wall surrounding a body opening is known from US 2019/0232036 A1. According to this, a minimally invasive device is used to obtain haemostasis of the tissue of a prostate capsule after a tissue resection, which is also performed minimally invasively in this capsule. The device essentially consists of a catheter, a distal balloon and a proximal balloon and can be inserted through the patient's bladder so that the distal balloon is in the bladder, immediately adjacent to the bladder neck, and the proximal balloon is inside the prostate capsule. Proximally, the inside of the prostate capsule should be sealed off from the ureter by the catheter running through it.

If the distal balloon is now filled with a pressurizing medium via a corresponding lumen that runs inside the catheter and thus inflated, the inside of the prostate capsule is also sealed distally against the bladder, with the distal balloon sealing against the neck of the bladder. The liquid haemostatic medium, which closes the wound, is then introduced into the resection cavity of the prostate capsule via a further lumen, whereby it is introduced into the resection cavity via an infusion opening located near the distal end of the catheter located inside the prostate capsule. In a further step, the proximal balloon is to be inflated via a further lumen in order to apply the haemostatic agent to the tissue wall and press it against the tissue wall via the compressive force transmitted by the proximal balloon.

In a further embodiment known in the present document, the liquid haemostatic fluid is to be supplied via the proximal balloon, the balloon being provided with pores evenly distributed around its circumference through which the haemostatic fluid flowing into the balloon is distributed inside the prostatic capsule. It is intended that the haemostatic fluid flowing into the balloon under pressure also ensures that the balloon expands and lies against the tissue wall in order to cause the haemostatic fluid to be evenly released at the tissue wall and pressed against it.

### SUMMARY OF THE INVENTION

It is an object of the present invention to develop a device for applying a therapeutic substance, which is in the form of a fluid, to a tissue wall surrounding a body orifice, in such a way that endoscopic treatment can be carried out with this device.

According to this, a device is provided by means of which a therapeutic medium in the form of a fluid can be applied to a tissue wall surrounding a body cavity. The device is arranged on a catheter which can be inserted into the body cavity and, after insertion, can be expanded radially in relation to the catheter to touch the tissue wall. The fluid supplied via the catheter reaches the tissue wall via fluid outlet openings.

Accordance to the invention, the catheter is to be designed as a tube of an endoscopic device, the tube accommodating the device at its distal end. Preferably, this endoscopic device is intended for treatments in the upper or lower gastrointestinal tract. The tube has a lumen in which a mechanical control element extends. By means of an actuating element, which engages proximally on the control element and can be actuated manually, longitudinal movements of the control element are effected, by means of which the device can be adjusted between a state of smallest radial extension and a state of largest radial extension.

The device has the outlet openings for the fluid at least in its section which touches the tissue wall in the expanded state. By moving the device longitudinally in a proximal direction, preferably by means of a corresponding movement of the tube, the therapeutic substance emerging from the outlet openings can be applied to a designated area of the tissue wall, which may be the gastric or intestinal mucosa. The substance can penetrate this area.

In contrast, according to US 2019/0232036 A1, which forms the generic term, a minimally invasive treatment of the prostate is provided, wherein the proximal balloon guided on a short catheter is inflated by means of a gaseous or liquid pressurizing medium to apply the therapeutic substance and to apply a force to the area of a resection wound. According to one embodiment, the substance can also be supplied via pores of the proximal balloon, whereby the opening cross-section of the pores increases when the balloon is inflated; however, this does not allow the quantity supplied and the distribution of the therapeutic substance to be controlled to a sufficient level.

In a further embodiment of the invention, the fluid is to be a haemostatic medium for producing haemostasis on the tissue wall after a tissue resection performed on the latter. Such a tissue resection performed endoscopically by means of a cutting tool can, for example, be carried out in the gastrointestinal tract. The endoscopic device, which has the device for applying the haemostatic agent, can be used by the doctor carrying out the treatment to reach remote operated areas and apply the haemostatic agent to the injured tissue in order to stop the bleeding that occurs. The haemostatic substance can be a fibrin glue, for example. The endoscope having a working channel can remain in the patient's body after the tissue resection, and the device according to the invention is inserted into the working channel together with the tube and the control element, which is preferably designed as a rope or wire

Furthermore, the device retracted into the lumen should be radially resiliently pretensioned in this state and, after it has emerged from the lumen, for which it can be displaced in the distal direction by means of the control element, move into its radially expanded position. With the device according to the invention retracted into the lumen, the tube is advanced in the working channel until its distal end, which protrudes from the working channel, is positioned at the point of the body orifice to be treated. When the device is then actuated in the distal direction and emerges from the distal end of the tube, it springs open radially and rests with its end sections against the tissue wall in order to apply the liquid therapeutic agent to it.

According to one embodiment of the invention, the device is formed by micro wires which, in their pushed-out state from the lumen, run towards each other forming an overall goblet-like contour. The micro wires can be shaped and pretensioned in such a way that, in the relaxed state, they move in a course with an external radius in which the distal ends lie against the tissue. Alternatively, it can also be a course with an internal radius in which a section of its distal ends comes into contact with the tissue. In this case, supply channels can run inside the micro wires, the outlet openings of which serve to apply the fluid to the tissue wall. The outlet openings are positioned in the micro wires in such a way that the agent is applied directly to the area to be treated. Alternatively, supply tubes can be fixed to the micro wires, each of which is provided with at least one outlet opening for applying the fluid to the tissue wall.

Furthermore, it is planned that the supply channels of the micro wires or the supply tubes are connected to the tube surrounding the control element or to a main tube surrounding the control element and displaceably guided within the lumen. At least one further lumen is provided in the tube, via which the device is supplied with the therapeutic substance. Alternatively, there may be a corresponding lumen in the main tube from which the micro wires and/or supply tubes extend.

In a further embodiment of the device according to the invention, the micro wires can be connected to each other via a double-walled foil arrangement, whereby the fluid is guided between the two foils and can be transferred to the tissue wall via outlet openings provided in the outer foil. In this case, the agent is guided between the two film layers and exits the intermediate space via the outlet openings of the outer film layer. This allows the agent to be applied to the tissue wall in a favourable manner and the tensioned double-layer film can exert pressure on the tissue wall. Alternatively, the film, which has the shape of an inverted funnel, can be designed as a single layer and merge at its edge into a bead connected to the catheter. In this case, the outlet openings for the haemostatic agent are located in the bead and in the funnel-like part of the foil, which is supplied via the lumen of the catheter and is guided from the funnel-shaped foil forming a base to the outlet openings of the bead, but can also exit at the latter. The shape of the bead and thus the pressure on the tissue wall can be changed by the mechanical control element that engages in the middle of the base.

Furthermore, according to an embodiment of the device according to the invention, the haemostasis substance can be supplied to the device via the lumen or a conduit running in the tube, whereby the device is formed by a sleeve-like foil. This is provided with the outlet openings for the fluid and connects partial sections of the catheter, which can be moved relative to each other in the axial direction by means of the control element. The control element causes a deformation of the sleeve-like foil arranged between the tube sections, which forms a bead-like shape radially outwards and exerts pressure on the tissue wall, whereby the bleeding can be stopped.

It is also provided that the control element is designed as a wire or cable. With this wire or cable, which is guided in the lumen and connected proximally to an actuating element, the device according to the invention, which is at a great distance from the actuating element to be operated by the doctor providing treatment, can be activated by technically simple means.

Finally, the device formed together with the tube for applying the substance in the form of a fluid can be designed for placement in a working channel of a medical endoscope. After an endoscopic examination of the gastrointestinal tract and any subsequent resection of polyps by means of the endoscope already positioned in the gastrointestinal tract, the device can be guided into the area of the body cavity in which a medical agent is to be administered or haemostasis is to be performed.

The invention is not limited to the specified combination of the features of the claimed invention. In addition, there are further possibilities of combining individual features with one another, in particular if they arise from the patent claims, the advantages stated in relation to these, the following description of the embodiments or directly from the drawing. Furthermore, the reference of the patent claims to the drawing by the use of reference signs is in no way intended to limit the scope of protection of the patent claims to the examples of embodiments shown.

The various features of novelty which characterize the invention are pointed out with particularity in the claims annexed to and forming a part of the disclosure. For a better understanding of the invention, its operating advantages, specific objects attained by its use, reference should be had to the drawings and descriptive matter in which there are illustrated and described preferred embodiments of the invention.

### BRIEF DESCRIPTION OF THE DRAWING

FIG. 1 is a schematic representation of a longitudinal section through a device according to the invention, which is arranged at the distal end of a tube, according to a first embodiment example, wherein the device is formed by micro wires and feed tubes running in a goblet-like manner to one another,

FIG. 2 a further design example as a schematic representation in longitudinal section, in which the micro wires are connected to each other via a double-walled foil arrangement,

FIG. 3 an alternative embodiment in which a foil forms a base with a circumferential bead and outlet openings are provided in the bead,

FIG. 4 a further design example as a schematic representation in longitudinal section, in which the device is formed by a sleeve-like foil connecting partial sections of the tubus, the partial sections being moved towards each other in the axial direction by means of the control element, so that the foil is formed radially outwards in a bead-like manner,

FIG. 5 a longitudinal section through a body cavity of a human body, in which a mucosa and other underlying tissue layers are injured as a result of a resection, a device according to the invention being arranged in the body cavity, in which micro wires running in a calyx-like manner apply a fluid to the injured tissue wall.

FIG. 6 a side view of a proximal end of a medical endoscope which accommodates an endoscopic device and

FIG. 7 enlarged to scale, a perspective view of the distal end of the medical endoscope of FIG. 6, in the working channel of which the endoscopic device according to the invention is arranged in accordance with the embodiment example according to FIG. 1

### DETAILED DESCRIPTION OF THE INVENTION

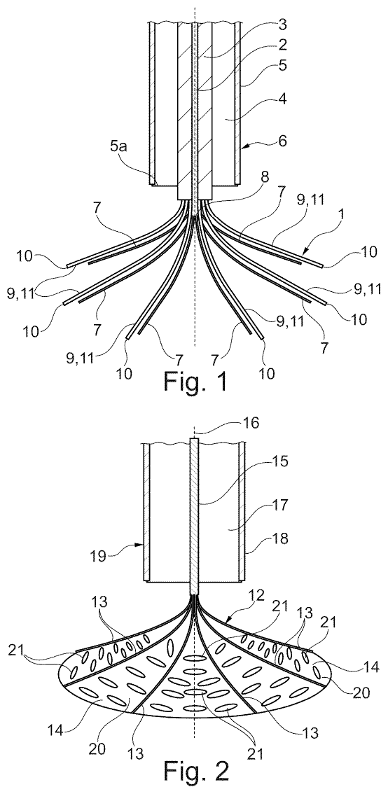

In FIG. 1, 1 refers to a device for applying a liquid therapeutic agent to a tissue wall. The tissue wall encloses a body cavity, as will be explained below in connection with the further FIG. 5. A human organ can also be adjacent to the body cavity.

The device 1 is connected to a control element 2 in the form of an actuating wire or cable and is enclosed by a main hose 3, whereby the main hose 3 runs in a lumen 4, which is formed in a tube 5 of an endoscopic device 6. The device 1 according to FIG. 1 consists of micro wires 7, which are radially resilient and are fixed to the control element 2 with their proximal ends 8. In the illustration according to FIG. 1, the micro wires 7 are in a state in which they are pushed out distally from the lumen 4 and spread out like a goblet. If the micro wires 7 are pulled into the tube 5 by means of the control element 2, they rest against an edge 5a of a distal end of the tube 5 and are pulled together radially.

A feed tube 9 is fixed to each of the individual micro wires 7, each of which has a supply channel 11 in its interior that ends distally with at least one outlet opening 10. Proximally, the feed tubes 9 are connected to the main hose 3, the latter being provided with a fluid channel, not shown in detail, via which the liquid agent enters the individual feed channels 11 in order to emerge from the distal ends of the feed tubes 9.

In a second embodiment example of a device 12 according to FIG. 2, this also consists of micro wires, which are designated with 13 and between which a double-walled foil 14 extends. The liquid substance is supplied to an intermediate space formed between the two layers of the double-walled foil 14 via a main hose 15, the main hose 15 being provided for this purpose with at least one fluid channel not shown in detail. Furthermore, a control element 16, which can also be designed as an actuating wire or cable, runs inside the main hose 15.

This control element 16 and a tube 18 receiving the main hose 15 via a lumen 17 are components of an endoscopic device 19. As can also be seen in FIG. 2, a proximally directed foil layer 20 has outlet openings 21 for the fluid, while a distally directed foil layer not visible in the illustration is closed. The device 12, i.e. the micro wires 13 together with the double-walled foil 14, are shown outside the lumen 17, so that they have spread out like the form of a goblet. When the device 12 is drawn into the lumen 17, the micro wires 13 are bundled and the double-walled foil 14 is folded.

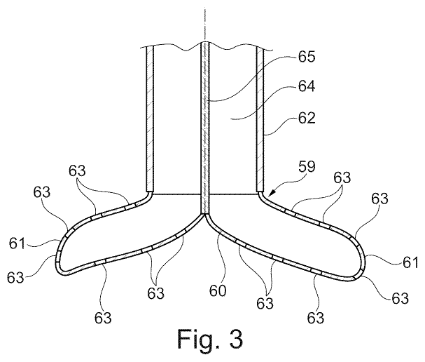

An alternative embodiment of a device 59 for applying a liquid therapeutic substance is shown in FIG. 3, in which a foil, which also has the shape of an inverted funnel in accordance with the foil 14 according to FIG. 2, forms a base 60 and is designed as a single layer. A bead 61 extends from the circumferential edge of this base 60 and is connected to the distal end of a tube 62. Outlet openings 63 for the haemostasis medium are formed both in the bead 61 and in the base 60. Via a lumen running inside the tube 62, the haemostasis medium is guided into a space 64 enclosed by the base 60 and the bead 61 and exits from this via the outlet openings 63. The shape of the bead 61 and thus the pressure on the tissue wall can be changed by a mechanical control element 65 acting centrally on the base 60.

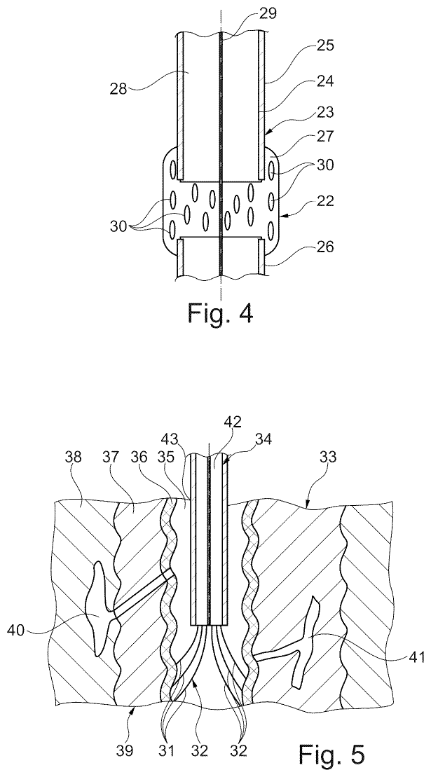

A third example of an embodiment of a device 22 according to the invention and an endoscopic device 23 adapted thereto is shown in FIG. 4. In this case, a tube 24 comprises a long proximal section 25 and a short distal end section 26, which are connected to one another by a sleeve-like foil 27. This overlaps the ends of the proximal section 25 and the distal end section 26 at their outer surfaces.

A control element 29 extending within a lumen 28 grips at least indirectly on an end of the distal end section 26, so that a tensile force transmitted to it by means of the control element 29 leads to a movement of the section 25 and the distal end section 26 towards each other in the axial direction. As a result, the foil 27 deforms radially outwards in a bead-like manner, so that pressure can be exerted on the tissue wall to be treated in order to deliver the therapeutic substance or haemostasis medium, which passes through outlet openings 30 provided in the foil 27 from the lumen 28 into the surface of the tissue and/or after a previous resection as a wound closure in the resulting wound in the tissue wall. As the sleeve-like film 27 expands radially like a bead, the therapeutic agent or haemostatic agent is pressed into the tissue wall to be treated.

In the illustration according to FIG. 5, an exemplary use of a device 31 is shown in a longitudinal section, in which, in contrast to the design example according to FIG. 1, separate feed tubes are dispensed with and instead feed channels not shown in more detail run inside micro wires 32. These are open at their distal ends. Part of the longitudinal section is also a part of a human organ, for example an intestine 33, into which the device 31 is inserted via an endoscopic device 34. The intestine 33 forms a body cavity 35 in its interior, which is enclosed by a tissue wall 39 consisting of different tissue layers 36, 37 and 38. The uppermost tissue layer 36 can therefore be an intestinal mucosa.

A resection has resulted in wounds 40 and 41 in some of these tissue layers 36, 37, 38, causing bleeding. By means of the endoscopic device 34, the device 31 is positioned at the point to be treated in the intestine 33 and the micro wires 32 are pushed out in the distal direction from a lumen 42 of a tube 43 of the endoscopic device 34, so that they spread open like a goblet and lie with their distal ends against the surface of the tissue wall 39 facing the body cavity 35. When the haemostatic drug supplied to the micro wires 32 via the tube 43 emerges distally from them, the entire device 31 is moved in a proximal direction, which leads to the application of the haemostatic drug to the tissue wall 39. This ensures that the haemostatic drug reaches the wounds 40 and 41, seals them and thus stops the bleeding that occurs.

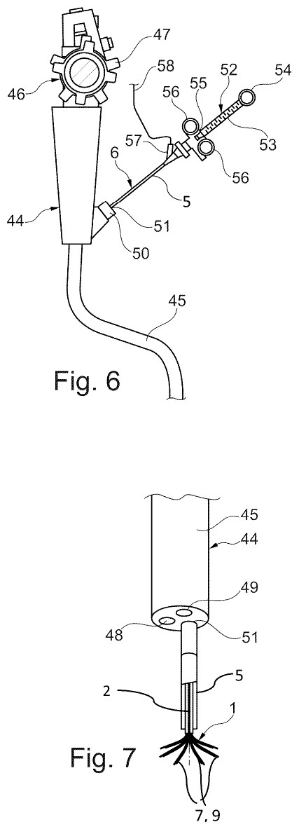

FIG. 6 shows a proximal section of a medical endoscope 44, with which, for example, examinations of the intestine can be carried out during a colonoscopy of the intestine and, if necessary, a polypectomy for resecting polyps can be carried out by means of an endoscopic device not shown guided in the endoscope 44. Only a proximal end section of this endoscope 44 is shown in FIG. 6, while FIG. 7, which is enlarged in scale compared to FIG. 6, shows a distal end of the medical endoscope 44.

According to FIG. 6, a control unit 46 connected to an insertion tube 45 is located at the proximal end and consists, among other things, of an angle control 47 with which the position of the distal end of the insertion tube 45 shown in FIG. 7, which accommodates a light source 48 and a camera 49, can be changed. Furthermore, a sluice 50 is located on the control unit 46, via which the device 1 according to the invention as shown in FIG. 1 can be inserted into a working channel 51 running inside the insertion tube 45. In the present case, a tube 5 as shown in FIG. 1 is inserted into the working channel 51 of the endoscope 44 via the sluice 50 and, as already explained, emerges from the distal end of the endoscope 44 as shown in FIG. 7. The tube 5 with the control element 2 is shown shortened, as is the insertion tube 45.

An actuating element 52 is arranged at a proximal end of the tube 5, which has a thumb ring 54 supported on the tube 5 via a guide element 53. Furthermore, a guiding bush 55 is longitudinally displaceably guided on the guiding element 53, to which finger rings 56 are fixed. The guiding bush 55 engaged with the control element 2 so that the control element 2, which runs inside the tube 5 as shown in FIG. 1, can be adjusted. Consequently, the displacement of the guiding bush 55 via the finger rings 56 can cause the device according to the invention with its micro wires 7 and feed tubes 9 to be displaced out of or into the lumen 4.

Using the finger rings 56 arranged on the guiding bush 55, the treating doctor can adjust the device 1 according to the invention with one hand, whereby his thumb engages in the thumb ring 54. A sluice 57 extends from the endoscopic device 6, to which a hose 58 for supplying the fluid is connected.

In FIG. 7, the device 1 shown in FIG. 1 is only shown as an example. It can also be one of the devices 12, 21, 31 or 59 shown in FIG. 2, 3 or 4 respectively.

While specific embodiments of the invention have been shown and described in detail to illustrate the inventive principles, it will be understood that the invention may be embodied otherwise without departing from such principles.

We claim:

1. A device for applying a therapeutic substance in the form of a fluid to a tissue wall which surrounds a body cavity, wherein the device is arranged on a catheter which can be inserted into the body cavity and, after insertion, can be expanded radially with respect to the catheter to touch the tissue wall, and the fluid supplied via the catheter reaches the tissue wall via outlet openings, wherein the catheter is designed as a tube of an endoscopic device, wherein the tube accommodates the device at its distal end, in that the tube has a lumen in which a mechanical control element extends, by means of the longitudinal movements of which, effected proximally via an actuating element, the device can be moved between one end of the tube and the other end of the tube can be adjusted between a state of minimum radial extension and a state of maximum radial extension, and in that the device has the outlet openings for the fluid at least in its section which contacts the tissue wall in the spread-open state.

2. The device according to claim 1, wherein the fluid is a haemostatic substance for achieving haemostasis on the tissue wall after a tissue resection performed thereon.

3. The device according to claim 1, wherein the device drawn into the lumen is radially spring-biased in this state and after its exit from the lumen, for which it is displaceable in the distal direction by means of the control element, moves into its radially expanded position.

4. The device according to claim 3, wherein the device is formed by micro wires which, when pushed out of the lumen, extend towards each other forming an overall cup-like contour.

5. The device according to claim 4, wherein feed channels run inside the micro wires, the outlet openings of which serve to apply the fluid to the tissue wall.

6. The device according to claim 4, wherein feed tubes for applying the fluid to the tissue wall are fixed to the micro wires, the outlet openings of which serve to apply the fluid to the tissue wall.

7. The device according to claim 5, wherein the supply channels of the micro wires or the supply tubes are connected to the tube surrounding the control element or to a main hose which is displaceably guided within the lumen and surrounds the control element.

8. The device according to claim 4, wherein the micro wires are connected to one another via a double-walled foil arrangement, the fluid being guided between the two foil layers and being transferable to the tissue wall via the outlet openings provided in the outer foil layer.

9. The device according to claim 1, wherein the fluid can be supplied to the device via the lumen or a supply conduit arranged therein, the device being formed by a sleeve-like foil which is provided with outlet openings for the fluid and connects together partial sections of the tube which can be moved relative to one another in the axial direction by means of the control element.

10. The device according to claim 1, wherein the control element is designed as a wire or cable.

11. The device according to claim 1, wherein the device is configured together with the endoscopic device for applying the substance formed as a fluid for an arrangement in a working channel of a medical endoscope.
